(19) United States
(12) Patent Application Publication
(10) Pub. No.: US 2003/0083323 A1
(43) Pub. Date: May 1, 2003

(54) Oral use of dehydroepiandrosterone and some of its derivatives as pigmentation regulators

(76) Inventors: Olivier De La Charriere, Paris (FR); Stephanie Nouveau, Boulogne Billancourt (FR); Francoise Forette, Paris (FR); Etienne Emile Baulieu, Neuilly Sur Seine (FR)

Correspondence Address: ALSTON & BIRD LLP, BANK OF AMERICA PLAZA, 101 SOUTH TRYON STREET, SUITE 4000, CHARLOTTE, NC, 28280-4000, US

(21) Appl. No.: 10/169,964

(22) PCT Filed: Jan. 10, 2001
(86) PCT No.: PCT/FR01/00062

(30) Foreign Application Priority Data
Jan. 12, 2000 (FR) 00 00321

(51) Int. Cl. A61K 7/021; A61K 31/56

(52) U.S. Cl. 514/177; 424/63

(57) ABSTRACT

The invention concerns the use of at least a compound selected in the group consisting of dehydroepiandrosterone, its precursors and its metabolic derivatives, for preparing a composition for oral administration and for use in regulating human skin and/or skin appendages.

The present invention relates to the use of dehydroepiandrosterone, of its precursors and/or of its metabolic derivatives, for producing a composition suitable for oral administration and intended to be used in regulating pigmentation.

The color of human skin varies depending on various factors, in particular on the seasons of the year, on ethnic origin and on sex. It is mainly determined by the nature and concentration of melanin produced by melanocytes.

In addition, at various times of life, some individuals experience the appearance on the skin, in particular on the hands and the neck and shoulders, of marks which are darker and/or more colored, giving the color of the skin a certain heterogeneity. These marks are also due to a considerable concentration of melanin in the keratinocytes located at the surface of the skin.

The mechanism of formation of skin pigmentation, i.e. of the formation of melanin, is particularly complex and involves, schematically, the following main steps:

Tyrosine→Dihydroxyphenylalanine(Dopa)→Dopaquinone→Dopachrome→Melanin

Tyrosinase (monophenol dihydroxyl phenylalanine or oxygen oxidoreductase EC 1.14.18.1) is the essential enzyme involved in this reaction sequence and acts only when it is in the mature state, under the action of certain biological factors. This enzyme in particular catalyzes the reaction for the conversion of tyrosine into Dopa by virtue of its hydroxylase activity and the reaction for the conversion of Dopa into dopaquinone by virtue of its oxidase activity.

In the epidermis, the melanocyte is involved in the epidermal melanin unit which comprises a melanocyte surrounded by approximately 36 neighboring keratinocytes. All individuals, regardless of phototype, have approximately the same number of melanocytes per given area of skin. Ethnic differences, in terms of pigmentation, are not due to the number of melanocytes, but to the properties of their melanosomes. Melanosomes are aggregated in complexes and are small in size. They are highly specialized organelles, the only function of which is melanin production. They are produced from the endoplasmic reticulum in the form of spherical vacuoles called premelanosomes. These premelanosomes contain an amorphous protein substrate, but no melanogenic enzymes. During maturation of the premelanosome, the amorphous substrate is organized into a fibrillar structure oriented along the longitudinal axis of the melanosome. Four stages of melanosome development are distinguished, corresponding to the intensity of melanization. The melanin is deposited uniformly over the internal fibrillar network of the melanosome and the opacity of the organelle increases until saturation is obtained. As the melanin is synthesized in the melanosomes, they move from the perinuclear region to the end of the dendrites of the melanocytes. Via phagocytosis, the end of the dendrites is captured by keratinocytes, the membranes are degraded and the melanosomes are redistributed in the keratinocytes.

Although the melanin level varies from one population to another, the amount, of tyrosinase does not vary significantly and the tyrosine messenger RNA level is identical in white skin or black skin. The variations in melanogenesis are therefore due to variations either in tyrosine kinase activity or in the ability of the keratinocytes to phagocytose melanosomes.

It is known that, in most populations, obtaining a brown coloration of the skin and maintaining a constant hair color are important aspirations.

On the other hand, in other cases, some individuals wish to lighten their body hair when it is dark, so that it is less visible.

Some diseases are also responsible for abnormal pigmentation of the skin, which it is desirable to correct. Mention may be made, in particular, of:

regional hyperpigmentations, related to melanocyte hyperactivity, such as idiopathic melasmas occurring during pregnancy (“pregnancy mask” or chloasma) or during estroprogestative contraception,

localized hyperpigmentations caused by benign melanocyte hyperactivity and proliferation, such as senile pigmentation marks known as actinic lentigo,

accidental hyperpigmentations or depigmentations, possibly due to photosensitization or to postlesional cicatrization,

certain leukodermias, such as vitiligo, an autoimmune disease characterized by the appearance on the skin of white plaques related to depigmentation,

canities (hair turning white) which is thought to be related to an autoimmune disease, in particular to vitiligo (R. P. R. Dawber in “Science des traitements capillaires” [Science of hair treatments] C. Zviak, publisher Masson, November 1987.

In the prior state of the art, topical pro-pigmenting agents have been proposed, either in the domain of artificial coloration by introducing exogenous colorants, such as DHA (dihydroxyacetone), supposed to give the skin and/or the superficial body growths a coloration as close as possible to the natural coloration, or in the domain of natural coloration, by stimulation of the natural pathways of pigmentation, for example using compounds which stimulate melanogenesis with or without the action of UV radiation, such as, for example, α-MSH (α-melanotropin) or prostaglandins.

In certain cases, if it is not possible to repigment the lesioned skin, the areas of residual normal skin are in the end depigmented so as to give the skin as a whole a homogeneous white shade.

A substance is recognized as being a depigmenting substance if it acts directly on the vitality of the epidermal melanocytes where melanogenesis takes place and/or if it interferes with one of the steps of melanin biosynthesis, either by inhibiting one of the enzymes involved in melanogenesis, or by intercalating as a structural analog of one of the chemical compounds of the chain of melanin synthesis, this chain then being blocked, leading to depigmentation.

The substances most commonly used as topical depigmenting substances are especially hydroquinone and its derivatives, in particular its ethers, such as the monomethyl ether and the monoethyl ether of hydroquinone. These compounds, which act directly by a cytotoxic effect on melanocytes, exhibit a definite effectiveness, but are not, unfortunately, free of side effects due to their cytotoxicity, which may make their use delicate, or even dangerous.

Thus, there remains a need for novel agents which regulate the pigmentation of human skin, of body hair and/or of hair, which can be used orally, and which are nontoxic and/or nonallergenic and easy to use.

Now, the applicant has found, unexpectedly, that dehydroepiandrosterone (DHEA), at least one of its precursors or at least one of its metabolic derivatives, administered orally, has a pigmentation-regulating effect, even at low concentrations, without showing toxicity.

DHEA is a natural steroid produced essentially by the adrenocortical glands. It is known for its antiaging properties related to its ability to promote epidermal keratinization (JP-07196467) and to combat osteoporosis (U.S. Pat No. 5,824,671). It is used in the treatment of dry skin, due to its ability to increase the endogenous production and secretion of sebum and to reinforce the barrier effect of the skin (U.S. Pat. No. 4,496,556), and also in the treatment of obesity and of diabetes (WO 97/13500) and in various diseases of hormonal origin, such as some cancers or osteoporosis (WO 94/16709). It has also been proposed to use DHEA sulfate against alopecia (JP-60142908) and for treating the various signs of aging, such as wrinkles, the loss of radiance of the skin and slacking of the skin (EP-0723775). However, none of those documents either describes or suggests the oral use of DHEA, of its precursors or of its metabolic derivatives, as agents which regulate the pigmentation of the skin and of the superficial body growths, which is the subject of the present invention. Consequently, the subject of the present invention is the use of at least one compound chosen from the group consisting of dehydroepiandrosterone, its precursors and its metabolic derivatives, for preparing a composition suitable for oral administration and intended to be used in regulating the pigmentation of human skin and/or of superficial body growths. In an advantageous embodiment of this use, said compounds are present in the composition as depigmenting agents for human skin.

In another advantageous embodiment of this use, said compounds are present in the composition as propigmenting agents for superficial body growths, in particular for body hair and/or for hair.

In another advantageous embodiment of this use, the precursor is chosen from the group comprising cholesterol, pregnenolone, 17α-hydroxypregnenolone, 5-androstenediol, dehydroepiandrosterone sulfate, 17α-hydroxypregnenolone sulfate and 5-androstenediol sulfate.

In another advantageous embodiment of this use, the metabolic derivative is chosen from the group comprising 5-androstene-3β-17β-diol (adiol), 5-androstene-3β-17β-diol sulfate and 4-androstene-3,17-dione.

In accordance with the use according to the present invention, the composition may be intended to improve the homogeneity of skin color.

In accordance with the use according to the invention, the composition may be a cosmetic composition or a dermatological composition, provided in all the pharmaceutical forms normally used for oral administration, in particular in the form of tablets which may or may not be breakable, of granules, of capsules, of gelatin capsules, of solutes, of suspensions or of solutions, and comprising at least one compound chosen from the group consisting of dehydroepiandrosterone, its precursors and its metabolic derivatives, as active principle, in combination with any suitable excipient.

In accordance with the use according to the present invention, the compounds are present in the composition in an amount which allows them to be administered at a dose of between 1 and 100 mg per day, preferably between 25 and 75 mg per day, said dosage being taken in one or more stages, for example with a unit dose of 50 mg.

The DHEA which can be used according to the invention is, for example, available from the company SIGMA or from the company AKZO NOBEL.

In accordance with the use according to the present invention, the compositions may be used in all cases in which repigmentation or depigmentation of the skin and/or of the superficial body growths is necessary.

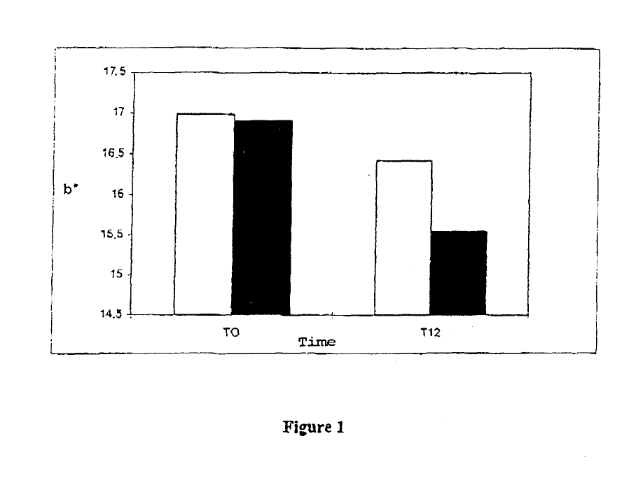

Other characteristics and advantages of the invention appear in the remainder of the description and the examples illustrated in FIG. 1.

FIG. 1 illustrates the depigmenting power of DHEA on human skin after 12 months of treatment at a dose of 50 mg/day according to example 1; □ placebo (n=69) ▪ DHEA 50 mg/day (n=66); (T0) corresponds to the start of treatment and (T12) to the end of treatment.

### EXAMPLE 1

Evaluation of the depigmenting power, on the skin, of DHEA administered orally—Comparison with a placebo

1. Methodology

The evaluation was carried out using a randomized double-blind clinical study on 140 individuals. The inclusion criteria were as follows: healthy female volunteers free of any severe pathological condition and 60 to 80 years old.

The treatment consisted, for 12 months, of a daily dose of 50 mg of DHEA, taken in the morning, in the form of a breakable tablet, for half the individuals, or of a placebo for the other half of the individuals.

The skin pigmentation is measured at the start of treatment (TO) and after 12 months (T12), by colorimetry using a Minolta CR200 chromameter (Minolta, 365, route de Saint-Germain 78424 Carrières sur Seine).

This machine describes the colors in a three-dimensional space, using the system of coordinates L, a* and b* and recommended by the CIE in 1976. In this system, L* expresses the luminance or clarity of the color (0 corresponding to black and 100 to white), a is the component for separation between green (negative) and red (positive) and b*, which varies from blue (negative) to yellow (positive) expresses the pigmentation of the skin. The latter parameter was used to evaluate the change in skin color during the treatment.

The machine is calibrated on a standard “white” (surface provided with the machine) at the beginning of each day of measurement and the calibration is controlled throughout the day.

The value of b* is the result of the mean of 6 individual measurements per measurement time. The statistical analysis is performed using the Wilcoxon test.

1. Results:

They are illustrated in FIG. 1.

After 12 months of treatment with DHEA at 50 mg per day, a significant decrease in pigmentation is observed compared to the placebo group (p=0.006, Wilcoxon test).

These results show that DHEA administered orally has a depigmenting effect on the skin.

### EXAMPLE 2

### Effect of oral DHEA on canities

1. Methodology:

The evaluation was carried out using a randomized double-blind clinical study on 140 individuals. The inclusion criteria were as follows: healthy male volunteers free of any severe pathological condition and 60 to 80 years old.

The treatment consisted, for 6 months, of a daily dose of 50 mg of DHEA, taken in the morning, in the form of a breakable tablet, for half the individuals, or of its placebo for the other half of the individuals.

At the end of the 6 months of treatment, the individuals answered a series of questions concerning the state of their hair and the effect of the treatment on various parameters, in particular regarding their degree of canities (improvement, worsening or no change in the number of white hairs).

The statistical analysis is performed with Wilcoxon tests.

2. Results:

They are given in table 1 below.

TABLE 1

| | Worsening | No change | Improvement | Total |
|---|---|---|---|---|
| DHEA | 0 | 59 | 5 | 64 |
| Placebo | 7 | 54 | 2 | 63 |

After 6 months of treatment with DHEA at the dose of 50 mg/kg/day, no individual experienced a worsening of his or her degree of canities, whereas 7 individuals experienced a worsening under placebo.

In addition, after treatment with DHEA at the dose of 50 mg/kg/day, 5 individuals experienced an improvement in their canities, i.e. the number of white hairs decreased.

The difference between the two treatments is significant (p=0.0094, Wilcoxon test).

These results show that DHEA administered orally has a propigmenting effect on hair.

1. The use of at least one compound chosen from the group consisting of dehydroepiandrosterone, its precursors and its metabolic derivatives, for preparing a composition suitable for oral administration and intended to be used in regulating pigmentation of human skin and/or of superficial body growths.

2. The use as claimed in claim 1, characterized in that said compounds are present in the composition as depigmenting agents for human skin.

3. The use as claimed in claim 1, characterized in that said compounds are present in the composition as propigmenting agents for superficial body growths.

4. The use as claimed in any one of claims 1 to 3, characterized in that the precursor is chosen from the group comprising cholesterol, pregnenolone, 17α-hydroxypregnenolone, 5-androstenediol, dehydroepiandrosterone sulfate, 17α-hydroxy-pregnenolone sulfate and 5-androstenediol sulfate.

5. The use as claimed in any one of claims 1 to 3, characterized in that the metabolic derivative is chosen from the group comprising 5-androstene-3β-17β-diol (adiol), 5-androstene-3β-17β-diol sulfate and 4-androstene-3,17-dione.

6. The use according to any one of claims 1 to 5, characterized in that the composition is intended to improve the homogeneity of skin color.

7. The use as claimed in any one of claims 1 to 6, characterized in that the composition is a cosmetic composition.

8. The use as claimed in any one of claims 1 to 6, characterized in that the composition is a dermatological composition.

9. The use as claimed in any one of claims 1 to 8, characterized in that the compounds are present in the composition in an amount which allows them to be administered at a dose of between 1 and 100 mg per day, preferably between 25 and 75 mg per day.
